(19) United States
(12) Patent Application Publication
(10) Pub. No.: US 2026/0091393 A1
(43) Pub. Date: Apr. 2, 2026

(54) APPARATUS AND METHOD OF AMPLIFYING SEGMENTS OF NUCLEIC ACID IN A MIXTURE SAMPLE VIA POLYMERASE CHAIN REACTION

(71) Applicant: University of Kentucky Research Foundation, Lexington, KY (US)

(72) Inventor: Guigen Zhang, Lexington, KY (US)

(21) Appl. No.: 19/320,813

(22) Filed: Sep. 5, 2025

Related U.S. Application Data
(60) Provisional application No. 63/691,275, filed on Sep. 5, 2024.
(60) Provisional application No. 63/691,695, filed on Sep. 6, 2024.

(51) Int. Cl. B01L 7/00; C12Q 1/686

(52) U.S. Cl. CPC B01L 7/525; C12Q 1/686; B01L 2300/0663; B01L 2300/1827

(57) ABSTRACT

An apparatus for amplifying segments of nucleic acid in a mixture sample via polymerase chain reaction (PCR) has a body including a heating element and a Mixture sample conduit. The heating element includes a denaturation zone, an extension zone and an annealing zone arranged on the body The Mixture sample conduit includes an inlet, an outlet and an intermediate section that passes serially through the denaturation zone, the annealing zone and the extension zone for multiple cycles. A related method for amplifying segments of nucleic acid in a mixture sample via polymerase chain reaction is also described.

### RELATED APPLICATIONS

This document claims the benefit of and priority to U.S. Provisional Patent Application Ser. No. 63/691,275, filed Sep. 5, 2024, and U.S. Provisional Patent Application Ser. No. 63/691,695, filed Sep. 6, 2024, the full disclosures of which are incorporated herein by reference.

### TECHNICAL FIELD

This document relates generally to an apparatus and method for conducting polymerase chain reactions and, more particularly, to a microfluidic-based, battery-operable (or low DC powered), and very low-cost apparatus made of a polymeric material to perform various types of polymerase chain reaction (PCR) for near-instantaneous DNA/RNA amplification.

### BACKGROUND

Since its invention in the 1980s, polymerase chain reaction (PCR) has become a technique essential in cellular and molecular biology that has played a pivotal role in improving the quality of life. Common examples include sequencing the human genomes, detection of infectious and genetic diseases, to forensic identifications, archaeologic and paleontological analyses, food and agricultural applications, etc. During the Covid-19 pandemic, we relearned that in identifying SARS-CoV-2 infections, PCR is still the most accurate and reliable method. A state-of-the-art PCR process is typically done by placing a mixture of prepared sample in a thermocycler. The mixture sample contains the DNA sample collected for analysis, primers, nucleotides (adenine (A), cytosine (C), guanine (G) and thymine (T)), Taq polymerase enzyme, and buffer to ensure the right conditions for the reactions.

Once inside the thermocycler, the mixture sample will go through three reaction steps repeatedly: (1) denaturation, in which double-strand DNA is separated to single strands through heating (typically at a temperature between 90° C. and 98° C.); (2) annealing, in which molecules are cooled such that specific primers and target DNA are complementary coupled according to base pairing principle (typically at a temperature between 50° C. and 65° C.); and (3) extension, in which individual nucleotides join the hybridization reaction to extend the primers by replicating the template or target DNA (typically at a temperature between 70° C. and 75° C.). Each of these three steps are repeated in the same order between 20 to 40 times with each successive cycle resulting in the doubling of the DNA copies.

At the heart of the state-of-the-art PCR is the thermocycler-an instrument that precisely controls and maintains the temperature by ramping it up and down from 90-98° C. to 50-65° C. and 70-75° C. to facilitate temperature-aided reactions. In the past several decades the method of performing PCR has seen generational improvements from the first-generation traditional method to second-generation quantitative PCR and third-generation digital PCR and digital droplet PCR. What has not changed, however, is its reliance on the thermocycler to provide the temperature change. Most conventional PCR utilizes either electric-coil-based or Peltier-based thermocyclers to control the solution temperature. Due to repeated heating and cooling in multiple cycles, the time spent on thermocycling for PCR, which includes the time for ramping up and down the temperature and the dwell time at each of these steps for the reaction to take place, can vary between one and four hours. While the dwell time at each reaction step may vary depending on the length (in base pair) of the target DNA, and the primers, enzyme and buffer used, in general it is about 3 or 4 times longer time at the extension step than those at the denaturation and annealing steps.

Many efforts have been made to shorten the time required for completing 30 to 40 cycles of PCR over the past decades. Typically, three approaches could be used to cut down the overall PCR time: on the biochemistry side, using enzymes and buffers that support fast reactions is pursued, and on the instrument side, either using a smaller sample volume or increasing the heating/cooling rate is often explored. For example, using a thin film heater deposited on a silicon wafer to provide 40-cycles of thermocycling to a 100 nL of 164-bp (base pair) long PCR mixture, Neuzil et el. [1] reported a under 6-minute PCR process. Wheeler et al. [2] developed thermal blocks from copper along with a porous foam and water baths, and completed 30-cycle to 5 mL of 58-bp long PCR mixture in less than 3 minutes. With a custom designed injection-molded microfluidic chips coupled with a pair of Peltier heating and cooling units, Nouwairi et al. [3] achieved 40-cycle 10 uL sample PCR in under 10 minutes. Moreover, using plasmon-excited Au film activated heating and cooling facilitated by turning on/off LED lights, Son et al. [4] reported completing 30-cycle 10 uL sample of 98-base long PCR in under 5 minutes. Lately, another photothermal method developed by Kim et al. [5] provided multiplexed 30-cycle 100 nL sample PCR in under 5 minutes.

Recently, a couple of fast PCR machines have also made it to the marketplace. For example, UK-based diagnostics company, LEX Diagnostics, provides a PCR machine to deliver sample to result within 5-7 minutes, and Molecular Biology Systems offers an ultrafast PCR system, NextGenPCR, which is said to perform a full 3 temperature, 30-cycle PCR in as little as 2 minutes for some fragments of DNA. PCR machines typically cost from $5,000 to over $100,000. While these new developments and products point to the possibility of shortening the time required to complete 30- or 40-cycle PCR to just few short minutes, they do not seem to help much in lowering the costs for PCR machines significantly, owing to the complex fabrication processes and components used.

This document relates to and describes an extremely low-cost apparatus that quite possibly provides the fastest and simplest solution to perform PCR.

### SUMMARY

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a loop”, as used herein, may also refer to, and encompass, a plurality of loops.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10 % of the stated numerical value

In accordance with the purposes and benefits set forth herein, a new and improved apparatus is provided for amplifying segments of nucleic acid in a mixture sample via polymerase chain reaction (PCR). That apparatus comprises, consists of or consists essentially of a body including a heating element and a mixture sample conduit. The heating element includes a denaturation zone, an extension zone and an annealing zone arranged on the body. The mixture sample conduit includes an inlet, an outlet and an intermediate section that passes serially through the denaturation zone, the annealing zone and the extension zone for multiple cycles.

In some of the many possible embodiments of the apparatus, the heating element includes a heating channel holding a conductive material and a power source adapted to apply a current to the conductive material whereby the conductive material acts as the heating element. In at least some embodiments, the heating element extends in what could be broadly described as a concentric ring pattern on the body. More specifically, the heating element runs continuously through segments of concentric semi-rings with interconnects between neighboring segments on the body. The three reaction zones may be arranged concentrically with the extension zone residing inside the annealing zone and the denaturation zone inside the extension zone.

In at least one of the many possible embodiments, the mixture sample conduit is oriented generally radially with respect to the heating element and the intermediate section includes a plurality of loops that extend serially through the denaturation zone, the annealing zone and the extension zone whereby each of the plurality of loops comprises a PCR cycle. In such an embodiment, the mixture sample, with the nucleic acid to be amplified, is delivered through the inlet to the intermediate section of the mixture sample conduit. Each loop of the plurality of loops making up the intermediate section includes (a) a cycle initiation section adapted for delivering the mixture sample to the denaturation section, (b) a denaturation dwell section adapted for maintaining the mixture sample in the denaturation zone for a first dwell time, (c) a first transition section adapted for delivering the mixture sample to the annealing zone, (d) an annealing dwell section adapted for maintaining the mixture sample in the annealing zone for a second dwell time, (e) a second transition section adapted for delivering the mixture sample to the extension zone and (f) an extension dwell section adapted for maintaining the mixture sample in the extension zone for a third dwell time. In some embodiments, the ratio of the first dwell time to the second dwell time to the third dwell time is about 1:1:3.

In at least some embodiments of the apparatus, the body includes a first section including the heating element and a second section including the mixture sample conduit. The first and second sections may interconnect or nest together. In one particularly useful embodiment, the body is disc shaped. In order to minimize costs in production, the body may be made from a polymeric material.

In at least some embodiments of the apparatus, an optic-spectrometer unit is connected to the outlet of the mixture sample conduit. Such an optic-spectrometer unit is adapted to enable in-situ spectroscopic reading of PCR results.

In at least some embodiments, the apparatus includes a mixture sample displacement device adapted to move the mixture sample through the mixture sample conduit from the inlet, through the intermediate section, where nucleic acid amplification of the nucleic acid sample is completed, to the outlet in a swift and efficient manner. The mixture sample displacement device may be a positive pressure generator connected to the inlet that is adapted to push the mixture sample through the mixture sample conduit. The mixture sample displacement device may be a negative pressure generator connected to the outlet that is adapted to draw or pull the mixture sample through the mixture sample conduit. In some embodiments, the mixture sample displacement device may be some combination of a positive pressure generator and a negative pressure generator.

In accordance with an additional aspect, a new and improved method is provided for amplifying segments of nucleic acid in a mixture sample via polymerase chain reaction (PCR). That method comprises, consists of or consists essentially of:
(a) displacing the mixture sample through a continuous mixture sample conduit including an inlet, an intermediate section and an outlet;
(b) serially guiding the mixture sample in a loop of the intermediate section through a denaturation zone, an annealing zone and an extension zone to complete a PCR cycle;
(c) repeating (b) a plurality of times to complete a plurality of PCR cycles; and
(d) discharging the mixture sample from the outlet.

In at least some embodiments, the method includes the additional step of forming the denaturation zone, the annealing zone and the extension zone with a continuous heating element. Such a heating element may extend in a concentric ring pattern or run continuously through segments of concentric semi-rings with interconnects between neighboring segments on the body. In at least one possible embodiment, the extension zone is concentrically oriented inside the annealing zone and the denaturation zone is concentrically oriented inside the extension zone. Of course, other structural arrangements are possible.

The method may further include the step of orienting the mixture sample conduit generally radially with respect to the heating element whereby a plurality of loops in the intermediate section extend serially through the denaturation zone, the annealing zone and the extension zone so that each of the plurality of loops comprises a PCR cycle.

The method may include providing each loop of the plurality of loops with (a) a cycle initiation section adapted for delivering the mixture sample to the denaturation section, (b) a denaturation dwell section adapted for maintaining the mixture sample in the denaturation zone for a first dwell time, (c) a first transition section adapted for delivering the mixture sample to the annealing zone, (d) an annealing dwell section adapted for maintaining the mixture sample in the annealing zone for a second dwell time, (e) a second transition section adapted for delivering the mixture sample to the extension zone and (f) an extension dwell section adapted for maintaining the mixture sample in the extension zone for a third dwell time.

In at least some of the many possible embodiments, the method includes pushing the mixture sample through the mixture sample conduit by applying a positive pressure at the inlet end. In other possible embodiments, the method includes drawing the mixture sample through the mixture sample conduit by applying a negative pressure at the outlet end. In still other possible embodiments, the method includes some combination of pushing the mixture sample through the mixture sample conduit by applying a positive pressure at the inlet end and drawing the mixture sample through the mixture sample conduit by applying a negative pressure at the outlet end.

In at least one of the many possible embodiments, the method includes (a) providing the heating element in a first body section, (b) providing the mixture sample conduit in a second body section, (c) interconnecting the first body section and the second body section to process a first mixture sample by the polymerase chain reaction; (d) disconnecting the first body section from the second body section; and (e) interconnecting a different second body section to the first body section to process a second mixture sample by the polymerase chain reaction.

In at least some embodiments, the method includes creating concentric denaturation, annealing and extension zones of desired temperatures by adjusting the width and spacing of the heating rings in each zone. Still further in at least some embodiments, the method includes providing a conductive material in a heating channel and applying a current to the conductive material whereby the conductive material acts as the heating element. In some embodiments, the method may include adjusting dwell times of the mixture sample in the denaturation, annealing and extension zones by adjusting a length of the mixture sample conduit in each of the denaturation, annealing and extension zones. In some embodiments, the method may include connecting an optic-spectrometer unit to the outlet of the mixture sample conduit to enable in-situ spectroscopic reading of PCR results.

In the following description, there are shown and described several different embodiments of the new and improved apparatus and method for amplifying segments of nucleic acid in a mixture sample via polymerase chain reaction. As it should be realized, the apparatus and method are capable of other, different embodiments and their several details are capable of modification in various, obvious aspects all without departing from the apparatus and method as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein by reference and forming a part of the specification, illustrate several aspects of the new and improved apparatus and method and together with the description serve to explain certain principles thereof.

FIG. 1 is a perspective view of one possible embodiment of the new and improved apparatus for amplifying segments of nucleic acid in a mixture sample via polymerase chain reaction.

FIG. 2 is a detailed top plan view of the heating element formed on a first section of the body of the apparatus illustrated in FIG. 1.

FIG. 3 is a detailed top plan view of the mixture sample conduit formed on a second section of the body of the apparatus illustrated in FIG. 1.

FIG. 4 is a perspective view of an alternative embodiment of the apparatus including an optic-spectrometer unit connected to the outlet of the mixture sample conduit to enable in-situ spectroscopic reading of PCR results.

FIG. 5 is a perspective view of yet another alternative embodiment of the apparatus modified for droplet PCR as well as multiplexing droplet PCR.

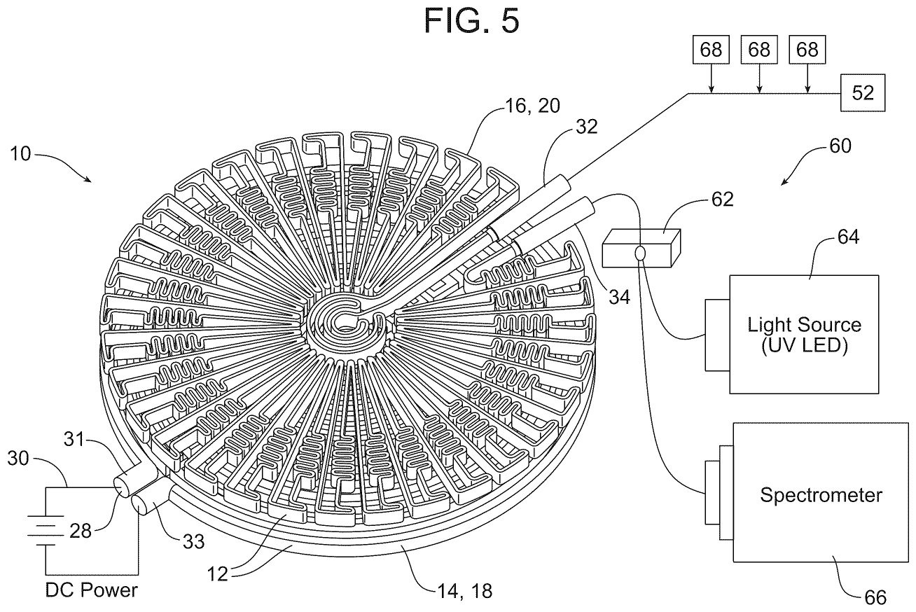

Reference will now be made in detail to the presently preferred embodiments of the apparatus and method for amplifying segments of nucleic acid in a mixture sample via polymerase chain reaction.

### DETAILED DESCRIPTION

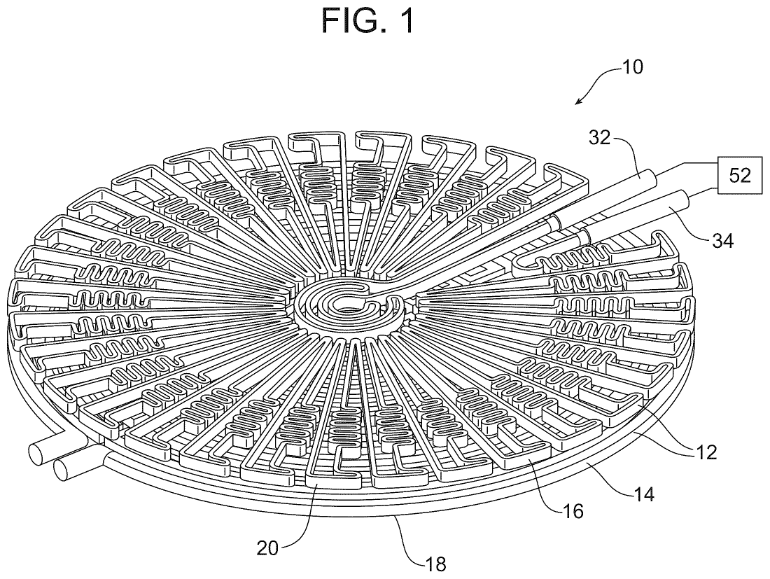

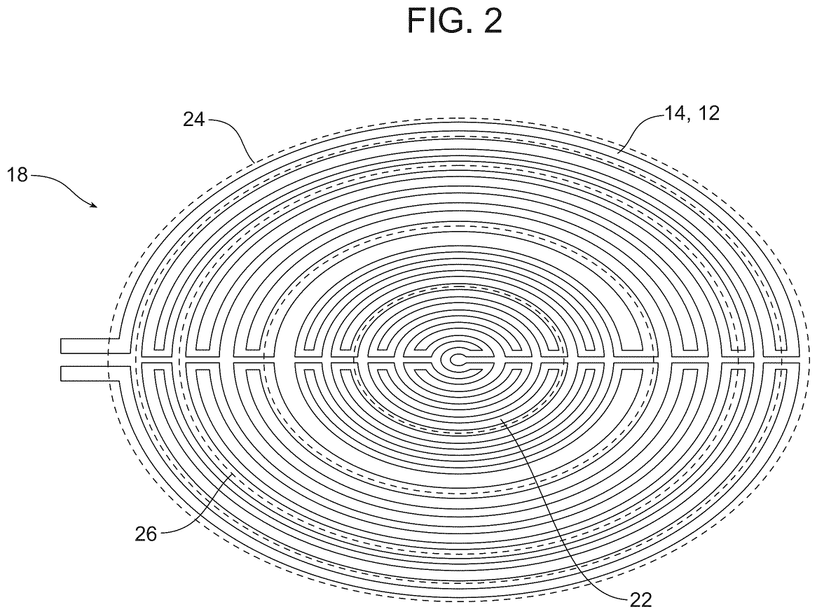

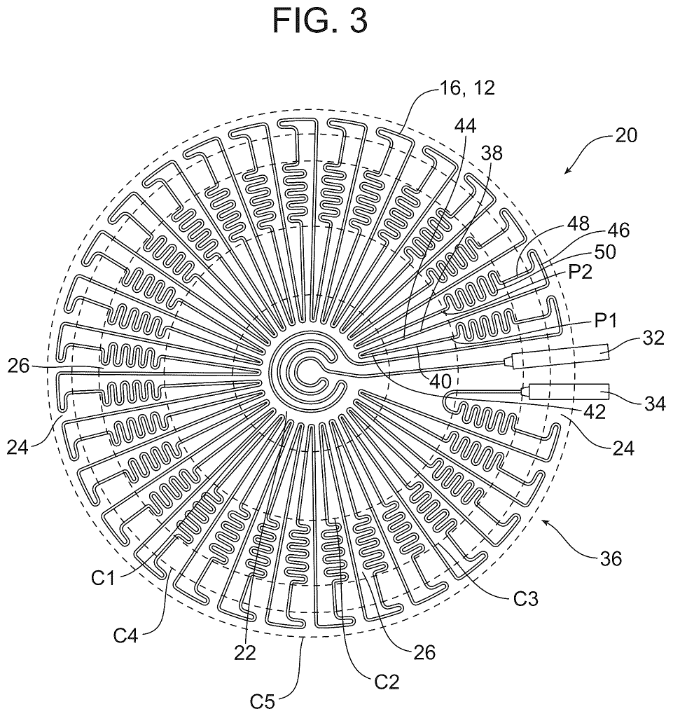

Reference is now made to FIGS. 1-3 that illustrate a first possible embodiment of the new and improved apparatus 10 for amplifying segments of nucleic acid in a mixture sample via polymerase chain reaction. That apparatus 10 includes a body 12 including a continuous heating element 14 and a cooperating, continuous mixture sample conduit 16. The body 12 may comprise a first section 18 and a second section 20. As best shown in FIG. 2, the first section 18 includes the heating element 14. As best shown in FIG. 3, the second section 20 includes the mixture sample conduit 16. As should be appreciated, the heating element 14 and the mixture sample conduit 16 are discrete from one another.

In the illustrated embodiment, the two body sections 18, 20 may be made from any appropriate material having the structural integrity to withstand the operating temperature range of the apparatus to promote PCR while also holding the mixture sample in the mixture sample conduit without dissolving, eroding or otherwise breaking down or altering the chemical makeup of the mixture sample. In some embodiments, the body 12, including the two sections 18, 20 may be made from a polymeric material to minimize production costs. Polymeric materials useful for this purpose include, but are not necessarily limited to, UV curing acrylate-based polymers such as poly(ethylene glycol)diacrylate, oxybis(methyl-2,1-ethanediyl)diacrylate, MiiCraft BV-007A, and other commonly used polymers for microfluidics including polydimethylsiloxane and SU8.

As illustrated in FIGS. 1-3, the body 12 may be formed in the shape of a disc and the two sections 18, 20 of the body may be connected, joined or nested together in any appropriate manner to allow for efficient heat transfer from the heating element 14 to the mixture sample conduit 16. In such an embodiment, the first section 18 with the heating element 14 may be reusable while the second section 20 with the mixture sample conduit 16 may be disposable, so that a new, pristine second section 20 and mixture sample conduit 16 is used for each new PCR application. In other embodiments, the body 12 may assume a different overall shape or even be made from a single unitary part that may be 3-D printed or constructed in some other appropriate manner known in the art.

The continuous heating element 14 of the illustrated embodiment, extends in a concentric ring pattern or runs continuously through segments of concentric semi-rings with interconnects between neighboring segments on the body or across the body 12/first body section 18, defining three distinct heating zones: (1) a denaturation zone 22, inside concentric dashed line circle C1, (2) an annealing zone 24, between concentric dashed line circles C4 and C5, and (3) an extension zone 26, between concentric dashed line circles C2 and C3. As illustrated, the extension zone 26 is concentrically oriented inside the annealing zone 24 and the denaturation zone 22 is concentrically oriented inside the extension zone. Of course, it should be appreciated that other architectures and zone layouts are possible.

In use, the denaturation zone 22 typically provides a mixture sample processing temperature of between about 91-96° C., the annealing zone 24 typically provides a mixture sample processing temperature of between about 58-63° C. and the extension zone 26 typically provides a mixture sample processing temperature of between about 71-79° C. Note that in some embodiments of reaction channel design, the denaturation zone 22 is divided into two sub-zones; the inner region is for initial heating and denaturation and the outer radially repeating region is for subsequent denaturation in each PCR cycle.

In the illustrated embodiment, the heating element 14 is a heating channel filled with an electrically and temperature conductive material 28 that serves as the heating element. Such a material may comprise a liquid metal, such as Galinstan, a nontoxic alloy composed of gallium, indium, and tin which melts at −19° C. Other metals of this type and conductive inks may also be used as the conductive material in the heating channel.

The heating element 14 may be designed by means of computational modeling using COMSOL. The width of each ring and the gap between rings of the heating element 14 are not uniform, as one can see in FIG. 2, in order to generate stable concentric zones 22, 24, 26 of desired temperatures for fast and efficient PCR processing of the mixture sample in the mixture sample conduit 16. Here, it should be noted that the heating element 14 has (a) the greatest density in the denaturation zone 22, which requires the highest operating temperature of about 91-96° C., (b) the lowest density in the annealing zone 24, which requires the lowest operating temperature of about 58-63° C. and an intermediate density in the extension zone 26, which requires an intermediate operating temperature of about 71-79° C. This design not only eliminates the use of expensive thermocyclers used in the state of the art PCR devices, but most importantly cuts out the time needed for repeatedly ramping the temperature up and down.

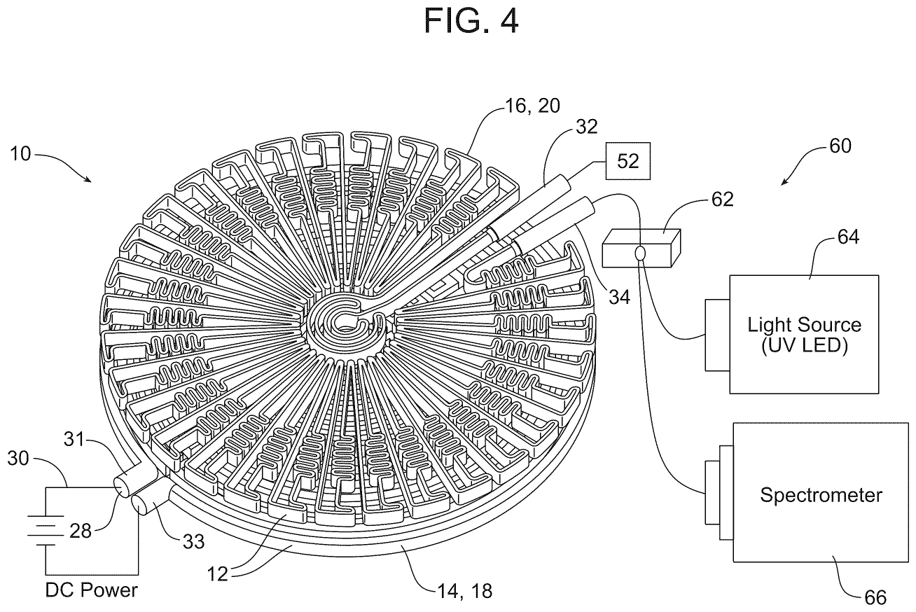

As shown in FIG. 4, a D.C. power source 30 (e.g. transformer, battery or other energy storage device such as a capacitor) is connected to the conductive material 28 at each end 31, 33 of the heating element 14 to cause resistance heating of the conductive material and generate the desired material sample processing temperatures in each of the three zones 22, 24, 26. Typically in order to generate the desired temperatures in the zones 22, 24, 26, a power in a range of between 1-5 W is provided at a DC voltage of between about 0.5V and 24.0V. The ratio of the first dwell time to the second dwell time to the third dwell time may vary from application to application but generally is about 1:1:3. Note that this ratio may be tuned slightly according to the specific target DNA/RNA, primers, and reagents, etc. used in the reactions.

As best illustrated in FIG. 3, the mixture sample conduit 16, includes an inlet 32, an outlet 34 and an intermediate section, generally designated by reference numeral 36, that passes serially through the denaturation zone 22, the annealing zone 24 and the extension zone 26 for multiple cycles. As shown in FIGS. 1 and 3, the mixture sample conduit 16 is oriented generally radially with respect to the heating element 14 and the intermediate section 36 includes a plurality of loops 38 that extend serially through the denaturation, annealing and extension zones 22, 24, 26 so that each loop comprises a complete PCR cycle. The illustrated embodiment includes thirty loops 38 so that the mixture sample passing completely through the mixture sample conduit 16 undergoes thirty PCR cycles. Here, it should be appreciated that the apparatus 10 could include substantially any number of loops from 1-n for processing the mixture sample for 1-n PCR cycles as required for any particular application.

Reference will now be made to the loop 38 shown in FIG. 3 which extends from the point P1 to the point P2. Each such loop 38 includes: (a) a cycle initiation section 40, extending between C2 and C1, adapted for initial denaturation and delivering the mixture sample to the denaturation section 22, (b) a denaturation dwell section 42, inside C1, adapted for maintaining the mixture sample in the denaturation zone for a first dwell time, (c) a first transition section 44, extending from C1 to C4, adapted for delivering the mixture sample to the annealing zone 24, (d) an annealing dwell section 46, extending between C4 and C5, adapted for maintaining the mixture sample in the annealing zone for a second dwell time, (e) a second transition section 48, extending from C4 to C3, adapted for delivering the mixture sample to the extension zone 26 and (f) an extension dwell section 50, extending between C3 and C2, adapted for maintaining the mixture sample in the extension zone for a third dwell time.

In the apparatus 10, nucleic acid amplification reactions are conducted by simply running the mixture sample through the length of the mixture sample conduit 16. As noted above, the mixture sample typically includes the nucleic acid sample (DNA or RNA) collected for analysis, primers, nucleotides (A (adenine), C (cytosine), G (guanine) and T (thymine) or U (uracil)), Taq polymerase enzyme, and buffer to ensure the right conditions for the reactions. The mixture sample is injected into the mixture sample conduit 14 at the inlet 32. A mixture sample displacement device 52, of a type known in the art, is then used to smoothly move the mixture sample from the inlet 32, through the intermediate section 36, including all of the loops 38 for completing all of the PCR cycles, and then through the outlet 34. The mixture sample displacement device may comprise, for example, a positive pressure generator connected to the inlet 32, a negative pressure generator connected to the outlet 34 or a combination of the two.

In an alternative embodiment of the apparatus 10 illustrated in FIG. 4, an optional optic-spectrometer unit 60 is connected to the outlet 34 of the mixture sample conduit 16. Such a unit 60 includes a sample chamber 62, a light source 64 (such as the UV LED light source shown, e.g., 365 nm to 405 nm, or other wavelength light sources suitable for the excitation of the relevant fluorescent dyes to be used) and a spectrometer 66. As the mixture sample, that has been subjected to the desired number of PCR cycles in the mixture sample conduit 16, passes from the outlet 34 into the sample chamber 62, an in-situ spectroscopic reading of the PCR results is provided by the unit 60. More specifically, the spectrometer 66 may be equipped with a photomultiplier to measure the intensity and wavelength of the excited fluorescence. Such an apparatus 10 could find wide-spread applications in research and analytic laboratories where small volume samples are typically handled, as well as in situations where large volume samples need to be processed. Moreover, the apparatus'simple, low-cost and battery-operable features, along with its unique capabilities to provide quantitative results at a near-instantaneous speed will help make quantitative PCR widely accessible and utilized even in remote areas

As should be clear from the above description, the apparatus 10 illustrated in FIG. 1-3 may be used to perform a method of amplifying segments of nucleic acid in a mixture sample via the polymerase chain reaction. Such a method may be broadly described as including the steps of: (a) displacing the mixture sample through a continuous mixture sample conduit 16 including an inlet 32, an intermediate section 36 and an outlet 34; (b) serially guiding the mixture sample in a loop 38 of the intermediate section through a denaturation zone 22, an annealing zone 24 and an extension zone 26 to complete a PCR cycle; (c) repeating (b) a plurality of times to complete a plurality of PCR cycles; and (d) discharging the now fully PCR processed mixture sample from the outlet 34.

The method may also include various other steps including, but not necessarily limited to any of the following, either alone or in any combination.
(1) forming the denaturation zone 22, the annealing zone 24 and the extension zone 26 with a continuous heating element 14;
(2) extending the heating element 14 in a concentric ring pattern whereby the extension zone 26 is concentrically oriented inside the annealing zone 24 and the denaturation zone 22 is concentrically oriented inside the extension zone;
(3) orienting the mixture sample conduit 16 generally radially with respect to the heating element 14 whereby a plurality of loops 38 in the intermediate section 36 extend serially through the denaturation zone 22, the annealing zone 24 and the extension zone 26 so that each of the plurality of loops comprises a PCR cycle;
(4) providing each loop 38 of the plurality of loops with (a) a cycle initiation section 40 adapted for delivering the mixture sample to the denaturation section 22, (b) a denaturation dwell section 42 adapted for maintaining the mixture sample in the denaturation zone for a first dwell time, (c) a first transition section 44 adapted for delivering the mixture sample to the annealing zone 24, (d) an annealing dwell section 46 adapted for maintaining the mixture sample in the annealing zone for a second dwell time, (e) a second transition section 48 adapted for delivering the mixture sample to the extension zone 26 and (f) an extension dwell section 50 adapted for maintaining the mixture sample in the extension zone for a third dwell time;
(5) pushing the mixture sample through the mixture sample conduit 16 by applying a positive pressure at the inlet end 32;
(6) drawing the mixture sample through the mixture sample conduit 16 by applying a negative pressure at the outlet end 34;
(7) (a) providing the heating element 14 in a first body section 18, (b) providing the mixture sample conduit 16 in a second body section 20, (c) interconnecting the first body section and the second body section to process a first mixture sample by the polymerase chain reaction; (d) disconnecting the first body section from the second body section; and (e) interconnecting a different second body section to the first body section to process a second mixture sample by the polymerase chain reaction;
(8) creating concentric denaturation, annealing and extension zones 22, 24, 26 of desired and stable (or steady-state) temperatures by adjusting a width and spacing (i.e. the density) of heating rings in each zone;
(9) providing a conductive material 28 in the heating channel and applying a current to the conductive material whereby the conductive material acts as a heating element 14;
(10) connecting an optic-spectrometer unit 60 to the outlet of the mixture sample conduit to enable in-situ spectroscopic reading of PCR results; and
(11) adjusting dwell times of the mixture sample in the denaturation, annealing and extension zones 22, 24, 26 by adjusting a length of the mixture sample conduit 16 in each of the denaturation, annealing and extension zones.

The apparatus 10 and related method described in this document relate to a microfluidic-based, optionally battery-operable, low-cost device to perform PCR for DNA or RNA amplification. This technology holds the potential for researchers to further shorten the reaction time and realize near-instantaneous PCR. The apparatus 10 and method advantageously allow for rapid medical diagnosis, rapid detection of infectious diseases as well as genetic and other diseases, timely forensic identification, food safety monitoring and value-added agricultural productions.

Although the apparatus and method of this disclosure have been illustratively described and presented by way of specific exemplary embodiments, and examples thereof, it is evident that many alternatives, modifications, or/and variations, thereof, will be apparent to those skilled in the art.

For example, by adding a series of droplet generators 68, of a type known in the art, upstream of the inlet 32 of the mixture sample conduit 16, the apparatus 10 may be provided with droplet PCR as well as multiplexing droplet PCR capabilities. To do that, a droplet channel layer containing multiple droplet generating sub-channels is added to the body 12. With this droplet layer, the collected DNA (or RNA) sample will be first divided into multiple portions. Each portion will be mixed, separately, with specifically selected primers designed for a different DNA/RNA target along with a different colored fluorescent dye, and other necessary components, and then partitioned into droplets suspended in an oil (e.g., mineral oil) using one of the droplet generators. All these prepared droplet samples suspended in oil will then be fed through the mixture sample conduit 16 for amplification reactions and optical reading. The same optical-spectroscopic unit 60 shown in FIG. 4 will be used since the photomultiplier used is able to detect fluorescence over a range of wavelengths. It is expected that a different target DNA/RNA will be identified by the accumulative intensity of the fluorescent signals at a wavelength corresponding to each of the selected fluorescent dyes used. For this multiplexing amplification purpose, precise control for the size of the droplets is not as important as for the uniformity of the droplets. So, the size of these droplets may vary from 1 um to sub-mm, but highly uniform or monodispersed droplets are much desirable.

Due to its fluidic nature, a multiplexing apparatus 10 of the type described can handle a large volume of sample, or a large number of droplets of sample. Because of this, the number of target DNA/RNA segments that can be amplified in a single PCR run will be limited either by our current genetic knowledge, or by the volume of the collected biological sample, or more precisely, by how many smaller portions can it be divided into without sacrificing PCR amplification quality.

The apparatus 10 may also be used for instantaneous droplet digital PCR. Droplet digital PCR (ddPCR) technique is based on partitioning of the sample into many micro-reactions in a well-defined volume (e.g., ˜1 nL or less) prior to amplification. After the PCR reaction, each droplet either does or does not contain the nucleic acid of interest, thus allowing estimation of the number of molecules in the reaction under the assumption of a Poisson distribution. The ddPCR technique provides absolute quantification of target DNA copies per input sample, possesses increased precision and sensitivity in detecting low target copies, and is relatively insensitive to potential PCR inhibitors.

It is particularly useful for low abundance targets, targets in complex backgrounds, allelic variants (e.g., SNPs) and for monitoring of subtle changes in target levels, as well as for viral load analysis and microbial quantification. However, a typical ddPCR machine can cost close to $100,000 dollars or more, making it less accessible by many researchers and clinics, and almost impossible for point-of-care or on-site applications.

An apparatus 10 equipped as described above with the droplet capability can be further refined to provide a low-cost yet highly precise ddPCR solution for a wide range of applications. To enable such a capability, one thing required on the instrument side is to ensure the droplet generators are capable of making uniform droplets with a precise volume of ˜1 nL or less, which translates to spherical droplets with a diameter of approximately 125 um or smaller.

It is intended that all such alternatives, modifications, or/and variations, fall within the spirit of, and are encompassed by, the broad scope of the appended claims.

What is claimed:

1. An apparatus for amplifying segments of nucleic acid in a mixture sample via polymerase chain reaction (PCR), comprising:
a body including a heating element and a mixture sample conduit wherein (a) the heating element includes a denaturation zone, an extension zone and an annealing zone arranged on the body and (b) the Mixture sample conduit includes an inlet, an outlet and an intermediate section that passes serially through the denaturation zone, the annealing zone and the extension zone for multiple cycles.

2. The apparatus of claim 1, wherein the heating element includes a heating channel holding a conductive material and a power source adapted to apply a current to the conductive material whereby the conductive material acts as the heating element.

3. The apparatus of claim 2, wherein the heating element extends substantially concentrically on the body wherein the extension zone is concentrically oriented inside the annealing zone and the denaturation zone is concentrically oriented inside the extension zone.

4. The apparatus of claim 3, wherein the Mixture sample conduit is oriented generally radially with respect to the heating element and the intermediate section includes a plurality of loops that extend serially through the denaturation zone, the annealing zone and the extension zone whereby each of the plurality of loops comprises a PCR cycle.

5. The apparatus of claim 4, wherein the mixture sample is delivered through the inlet to the intermediate section and each loop of the plurality of loops includes (a) a cycle initiation section adapted for delivering the mixture sample to the denaturation section, (b) a denaturation dwell section adapted for maintaining the mixture sample in the denaturation zone for a first dwell time, (c) a first transition section adapted for delivering the mixture sample to the annealing zone, (d) an annealing dwell section adapted for maintaining the mixture sample in the annealing zone for a second dwell time, (e) a second transition section adapted for delivering the mixture sample to the extension zone and (f) an extension dwell section adapted for maintaining the mixture sample in the extension zone for a third dwell time.

6. The apparatus of claim 5, wherein the ratio of the first dwell time to the second dwell time to the third dwell time is about 1:1:3

7. The apparatus of claim 3, wherein the body includes a first section including the heating element and a second section including the Mixture sample conduit.

8. The apparatus of claim 7, wherein the body is made from a polymeric material.

9. The apparatus of claim 3, further including an optic-spectrometer unit, connected to the outlet of the Mixture sample conduit, adapted to enable in-situ spectroscopic reading of PCR results.

10. The apparatus of claim 3, further including a mixture sample displacement device adapted to move the mixture sample through the Mixture sample conduit from the inlet, through the intermediate section to the outlet wherein the mixture sample displacement device is selected from a group of devices including a positive pressure generator connected to the inlet, a negative pressure generator connected to the outlet and a combination thereof.

11. A method of amplifying segments of nucleic acid in a mixture sample via polymerase chain reaction (PCR), comprising:
(a) displacing the mixture sample through a continuous Mixture sample conduit including an inlet, an intermediate section and an outlet;
(b) serially guiding the mixture sample in a loop of the intermediate section through a denaturation zone, an annealing zone and an extension zone to complete a PCR cycle;
(c) repeating (b) a plurality of times to complete a plurality of PCR cycles; and
(d) discharging the mixture sample from the outlet.

12. The method of claim 11, including forming the denaturation zone, the annealing zone and the extension zone with a continuous heating element.

13. The method of claim 12, including extending the heating element in a concentric ring pattern whereby the extension zone is concentrically oriented inside the annealing zone and the denaturation zone is concentrically oriented inside the extension zone.

14. The method of claim 13, including orienting the Mixture sample conduit generally radially with respect to the heating element whereby a plurality of loops in the intermediate section extend serially through the denaturation zone, the annealing zone and the extension zone so that each of the plurality of loops comprises a PCR cycle.

15. The method of claim 14, including providing each loop of the plurality of loops with (a) a cycle initiation section adapted for delivering the mixture sample to the denaturation section, (b) a denaturation dwell section adapted for maintaining the mixture sample in the denaturation zone for a first dwell time, (c) a first transition section adapted for delivering the mixture sample to the annealing zone, (d) an annealing dwell section adapted for maintaining the mixture sample in the annealing zone for a second dwell time, (e) a second transition section adapted for delivering the mixture sample to the extension zone and (f) an extension dwell section adapted for maintaining the mixture sample in the extension zone for a third dwell time.

16. The method of claim 15, including pushing the mixture sample through the Mixture sample conduit by applying a positive pressure at the inlet end.

17. The method of claim 15, including drawing the mixture sample through the Mixture sample conduit by applying a negative pressure at the outlet end.

18. The method of claim 14, including (a) providing the heating element in a first body section, (b) providing the Mixture sample conduit in a second body section, (c) interconnecting the first body section and the second body section to process a first mixture sample by the polymerase chain reaction; (d) disconnecting the first body section from the second body section; (e) interconnecting a different second body section to the first body section to process a second mixture sample by the polymerase chain reaction.

19. The method of claim 13, including creating concentric denaturation, annealing and extension zones of desired stable temperatures by adjusting a width and spacing of heating rings in each zone.

20. The method of claim 19, including providing a conductive material in a heating channel and applying a current to the conductive material whereby the conductive material acts as the heating element.

21. The method of claim 11, including connecting an optic-spectrometer unit to the outlet of the Mixture sample conduit to enable in-situ spectroscopic reading of PCR results.

22. The method of claim 14, including adjusting dwell times of the mixture sample in the denaturation, annealing and extension zones by adjusting a length of the Mixture sample conduit in each of the denaturation, annealing and extension zones.
